(19) United States
(12) Patent Application Publication
(10) Pub. No.: US 2024/0016442 A1
(43) Pub. Date: Jan. 18, 2024

(54) Technique For Identifying Mild Cognitive Impairment Based On Gaze Information

(71) Applicant: HAII corp., Seoul (KR)

(72) Inventors: Ho Yung KIM, Seoul (KR); Dong Han KIM, Seoul (KR); Hye Bin HWANG, Incheon (KR); Chan Yeong PARK, Seoul (KR); Ji An CHOI, Seoul (KR); Hyun Jeong KO, Seoul (KR); Su Yeon PARK, Seoul (KR); Byung Hun YUN, Seoul (KR)

(73) Assignee: HAII corp., Seoul (KR)

(21) Appl. No.: 18/316,032

(22) Filed: May 11, 2023

(30) Foreign Application Priority Data
Jul. 13, 2022 (KR) 10-2022-0086039

(51) Int. Cl. A61B 5/00; A61B 5/16

(52) U.S. Cl. CPC A61B 5/4088; A61B 5/163; A61B 5/7282

(57) ABSTRACT

A method for identifying mild cognitive impairment by at least one processor of an apparatus according to some embodiments of the present disclosure is disclosed. the method comprises performing a task of acquiring digital biomarker data for identifying mild cognitive impairment of a user of a user terminal; performing a preliminary task to allow the user to check the task before performing the task; and acquiring gaze information of the user in conjunction with performing the task and the preliminary task; wherein the gaze information is used when determining displayed information in the preliminary task, and used when identifying mild cognitive impairment of the user in the task.

### 1. TECHNICAL FIELD

The present disclosure relates to a technique for identifying mild cognitive impairment, more particularly to a device for identifying mild cognitive impairment using user's gaze information according to tests and a method thereof.

### 2. RELATED ART

Alzheimer's disease (AD), which is a brain disease caused by aging, causes progressive memory impairment, cognitive deficits, changes in individual personality, etc. In addition, dementia refers to a state of persistent and overall cognitive function decline that occurs when a person who has led a normal life suffers from damage to brain function due to various causes. In this case, cognitive function refers to various intellectual abilities, such as memory, language ability, temporal and spatial understanding ability, judgment ability, and abstract thinking ability. Each cognitive function is closely related to a specific part of the brain. The most common form of dementia is Alzheimer's disease.

Various methods have been proposed for diagnosing Alzheimer's disease, dementia, or mild cognitive impairment. For example, a method of diagnosing Alzheimer's disease or mild cognitive impairment using the expression level of miR-206 in the olfactory tissue, and a method for diagnosing dementia using a biomarker that characteristically increases in blood are known.

However, since special equipment or tests necessary for biopsy are required so as to use miR-206 in the olfactory tissue, and blood from a patient should be collected by an invasive method so as to use biomarkers in blood, there is a disadvantage that the patient's rejection feeling is relatively large.

Therefore, there is an urgent need to develop a method capable of diagnosing mild cognitive impairment in a way in which the patient hardly feels a sense of rejection without any special equipment or examination.

### SUMMARY

The present disclosure has been made in view of the above problems, and it is one object of the present disclosure to provide an accurate mild cognitive impairment diagnosis method where patients hardly feel rejection.

It will be understood that technical problems of the present disclosure are not limited to the aforementioned problem and other technical problems not referred to herein will be clearly understood by those skilled in the art from the description below.

In accordance with an aspect of the present disclosure, the above and other objects can be accomplished by the provision of a method of identifying mild cognitive impairment by at least one processor of a device, the method including: performing a task of acquiring digital biomarker data for identifying mild cognitive impairment of a user of a user terminal; performing a preliminary task to allow the user to check the task before performing the task; and acquiring gaze information of the user in conjunction with performing the task and the preliminary task.

In accordance with some embodiments of the present disclosure, the preliminary task may include: a first preliminary task causing a first object to be displayed on a first region of a screen displayed on the user terminal; a second preliminary task causing a second object to be displayed on a second region different from the first region on the screen of the user terminal instead of the first object; a third preliminary task of determining whether the user is gazing at a third region opposite to the second region based on the gaze information in conjunction with performing the second preliminary task; and a fourth preliminary task causing different information to be displayed on the screen of the user terminal based on whether the user is gazing at the third region.

In accordance with some embodiments of the present disclosure, the fourth preliminary task may include: a first information output task causing first information that the user is gazing at the third region to be displayed on the screen of the user terminal if it is recognized that the user's gaze is gazing at the third region based on the gaze information; or a second information output task causing second information prompting the user to gaze at the third region to be displayed on the screen of the user terminal when it is recognized that the gaze of the user does not gaze at the third region based on the gaze information.

In accordance with some embodiments of the present disclosure, the second information may be displayed in a region different from a region in which the first information is displayed on the screen of the user terminal.

In accordance with some embodiments of the present disclosure, the second information may be displayed on a peripheral region of the second region where the second object is displayed.

In accordance with some embodiments of the present disclosure, the task may include: a first task causing the first object to be displayed on the first region of the screen displayed on the user terminal after the fourth preliminary task is performed; a second task causing the second object to be displayed on the second region instead of the first object on the screen of the user terminal; and a third task of acquiring the digital biomarker data for identifying mild cognitive impairment of the user based on first gaze information acquired while performing the first task and the second task a preset number of times.

In accordance with some embodiments of the present disclosure, the method may further comprises calculating a score value by inputting the digital biomarker data into a pre-learned mild cognitive impairment identification model; and determining whether mild cognitive impairment exists based on the score value.

In accordance with some embodiments of the present disclosure, the gaze information may be generated by the device analyzing an image after the device receives the image including eyes of the user from the user terminal.

In accordance with some embodiments of the present disclosure, the gaze information may be information received by the device from the user terminal, and is generated by the user terminal analyzing an image including eyes of the user.

In accordance with some embodiments of the present disclosure, the digital biomarker data may comprise information on a number of times the user gazed at the third region, information on a number of times the user gazed at a region other than the third region, information on whether the user's gaze continues to stare at a specific point for the preset time, information on a time elapsed for the user's gaze to move while performing the second task, information on movement speed of the user's gaze and information on whether the user's gaze is accurately staring at the third region.

In accordance with another aspect of the present disclosure, a computer program stored on a computer-readable storage medium, wherein the computer program, when executed on at least one processor of a device, performs processes of identifying mild cognitive impairment, the processes comprising: performing a task of acquiring digital biomarker data for identifying mild cognitive impairment of a user of a user terminal; performing a preliminary task to allow the user to check the task before performing the task; and acquiring gaze information of the user in conjunction with performing the task and the preliminary task.

In accordance with yet another aspect of the present disclosure, there is provided a device for identifying mild cognitive impairment, the device comprising: a storage configured to store at least one program instruction; and at least one processor configured to execute the at least one program instruction, wherein the at least one processor performs a task of acquiring digital biomarker data for identifying mild cognitive impairment of a user of a user terminal, performs a preliminary task to allow the user to check the task before performing the task, and acquires gaze information of the user in conjunction with performing the task and the preliminary task.

The technical solutions obtainable in the present disclosure are not limited to the above-mentioned solutions, other solutions not mentioned will be clearly understood by those skilled in the art from the description below.

The effect of the technique for identifying the mild cognitive impairment according to the present disclosure will be described.

According to some embodiments of the present disclosure, the mild cognitive impairment can be accurately diagnosed in a way in which the user hardly feels objection.

It will be understood that effects obtained by the present disclosure are not limited to the aforementioned effect and other effects not referred to herein will be clearly understood by those skilled in the art from the description below.

### BRIEF DESCRIPTION OF THE DRAWINGS

Various embodiments of the present disclosure are described with reference to the accompanying drawings. In this case, like reference numbers are used to refer to like elements. In the following embodiments, numerous specific details are set forth so as to provide a thorough understanding of one or more embodiments for purposes of explanation. It will be apparent, however, that such embodiment(s) may be practiced without these specific details.

FIG. 1 is a schematic diagram for explaining a system for identifying a mild cognitive impairment according to some embodiments of the present disclosure.

FIG. 2 is a flowchart illustrating an example of a method of acquiring digital biomarker data according to some embodiments of the present disclosure.

FIG. 3 is a flowchart illustrating an example of a method of performing a preliminary task according to some embodiments of the present disclosure.

FIG. 4 is a flowchart illustrating an example of a method of outputting another message when performing a preliminary task according to some embodiments of the present disclosure.

FIG. 5 is a flowchart illustrating an example of a message displayed when performing a preliminary task according to some embodiments of the present disclosure.

FIG. 6 is a diagram for explaining an example of a screen displayed on a user terminal when a preliminary task or a task is performed according to some embodiments of the present disclosure.

FIG. 7 is a diagram for explaining an example of a method of acquiring gaze information according to some embodiments of the present disclosure.

FIG. 8 is a flowchart illustrating a process of performing a task of acquiring digital biomarker data according to some embodiments of the present disclosure.

FIG. 9 is a flowchart illustrating an example of a method of determining whether a user has mild cognitive impairment using digital biomarker data according to some embodiments of the present disclosure.

FIG. 10 is a diagram for explaining an example of information included in digital biomarker data according to some embodiments of the present disclosure.

### DETAILED DESCRIPTION

Hereinafter, various embodiments of an apparatus according to the present disclosure and a method of controlling the same will be described in detail with reference to the accompanying drawings. Regardless of the reference numerals, the same or similar components are assigned the same reference numerals, and overlapping descriptions thereof will be omitted.

Objectives and effects of the present disclosure, and technical configurations for achieving the objectives and the effects will become apparent with reference to embodiments described below in detail in conjunction with the accompanying drawings. In describing one or more embodiments of the present disclosure, a detailed description of known functions and configurations incorporated herein will be omitted when it may make the subject matter of the present disclosure unclear.

The terms used in the specification are defined in consideration of functions used in the present disclosure, and can be changed according to the intent or conventionally used methods of clients, operators, and users. The features of the present disclosure will be more clearly understood from the accompanying drawings and should not be limited by the accompanying drawings, and it is to be appreciated that all changes, equivalents, and substitutes that do not depart from the spirit and technical scope of the present disclosure are encompassed in the present disclosure.

The suffixes “module” and “unit” of elements herein are used for convenience of description and thus can be used interchangeably and do not have any distinguishable meanings or functions.

Terms including an ordinal number, such as first, second, etc., may be used to describe various elements, but the elements are not limited by the terms. The above terms are used only for the purpose of distinguishing one component from another component. Therefore, a first component mentioned below may be a second component within the spirit of the present description.

A singular expression includes a plural expression unless the context clearly dictates otherwise. That is, a singular expression in the present disclosure and in the claims should generally be construed to mean “one or more” unless specified otherwise or if it is not clear from the context to refer to a singular form.

The terms such as “include” or “comprise” may be construed to denote a certain characteristic, number, step, operation, constituent element, or a combination thereof, but may not be construed to exclude the existence of or a possibility of addition of one or more other characteristics, numbers, steps, operations, constituent elements, or combinations thereof.

The term “or” in the present disclosure should be understood as “or” in an implicit sense and not “or” in an exclusive sense. That is, unless otherwise specified or clear from context, “X employs A or B” is intended to mean one of natural implicit substitutions. That is, when X employs A; when X employs B; or when X employs both A and B, “X employs A or B” can be applied to any one of these cases. Furthermore, the term “and/or” as used in the present disclosure should be understood to refer to and encompass all possible combinations of one or more of listed related items.

As used in the present disclosure, the terms “information” and “data” may be used interchangeably.

Unless otherwise defined, all terms (including technical and scientific terms) used in the present disclosure may be used with meanings that can be commonly understood by those of ordinary skill in the technical field of the present disclosure. Also, terms defined in general used dictionary are not to be excessively interpreted unless specifically defined

However, the present disclosure is not limited to embodiments disclosed below and may be implemented in various different forms. Some embodiments of the present disclosure are provided merely to fully inform those of ordinary skill in the technical field of the present disclosure of the scope of the present disclosure, and the present disclosure is only defined by the scope of the claims. Therefore, the definition should be made based on the content throughout the present disclosure

In the present disclosure, it is assumed that mild cognitive impairment is a concept including dementia and will be described below.

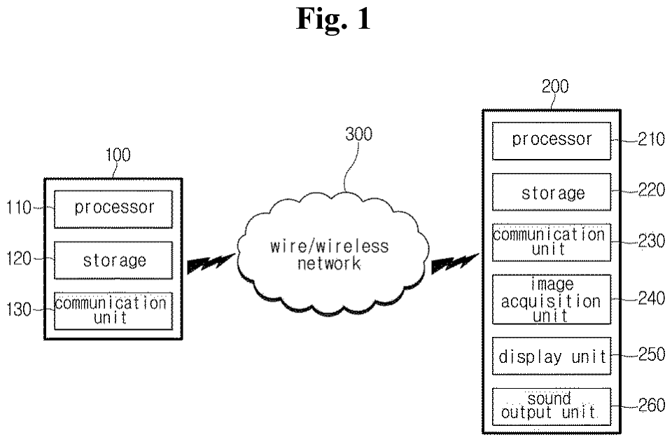

According to some embodiments of the present disclosure, at least one processor (hereinafter, referred to as ‘processor’) of the device may determine whether the user has mild cognitive impairment by using a mild cognitive impairment identification model. Specifically, the processor may acquire digital biomarker data by using the user's gaze information after acquiring gaze information. And, the processor may acquire a score value by inputting the digital biomarker data into the mild cognitive impairment identification model. Also, the processor may determine whether or not the user has mild cognitive impairment based on the score value. Hereinafter, a method for identifying mild cognitive impairment will be described with reference to FIGS. 1 to 10.

FIG. 1 is a schematic diagram for explaining a system for identifying a mild cognitive impairment according to some embodiments of the present disclosure.

Referring to FIG. 1, the system for identifying a mild cognitive impairment may include a device 100 for identifying mild cognitive impairment and a user terminal 200 for a user requiring identification of mild cognitive impairment. In addition, communication between the device 100 and the user terminal 200 may be connected through the wired/wireless network 300. However, the components constituting the system shown in FIG. 1 are not essential for implementing a system for identifying the mild cognitive impairment, and may have more or fewer components than the components listed above.

The device 100 of the present disclosure may be paired with or connected to the user terminal 200 over the wire/wireless network 300, thereby transmitting/receiving predetermined data. In this case, data transmitted/received over the wire/wireless network 300 may be converted before transmission/reception. In this case, the “wire/wireless network” 300 collectively refers to a communication network supporting various communication standards or protocols for pairing and/or data transmission/reception between the device 100 and the user terminal 200. The wire/wireless network 300 includes all communication networks to be supported now or in the future according to the standard and may support all of one or more communication protocols for the same.

The device 100 for identifying the mild cognitive impairment may include a processor 110, storage 120, and a communication unit 130. The components illustrated in FIG. 1 are not essential for implementing the device 100, and thus, the device 100 described in the present disclosure may include more or fewer components than those listed above.

Each component of the device 100 of the present disclosure may be integrated, added, or omitted according to the specifications of the device 100 that is actually implemented. That is, as needed, two or more components may be combined into one component or one component may be subdivided into two or more components. In addition, a function performed in each block is for describing an embodiment of the present disclosure, and the specific operation or device does not limit the scope of the present disclosure.

The device 100 described in the present disclosure may include any device that transmits and receives at least one of data, content, service, and application, but the present disclosure is not limited thereto.

The device 100 of the present disclosure includes, for example, any standing devices such as a server, a personal computer (PC), a microprocessor, a mainframe computer, a digital processor and a device controller; and any mobile devices (or handheld device) such as a smart phone, a tablet PC, and a notebook, but the present disclosure is not limited thereto.

In an embodiment of the present disclosure, the term “server” refers to a device or system that supplies data to or receives data from various types of user terminals, i.e., a client.

For example, a web server or portal server that provides a web page or a web content (or a web service), an advertising server that provides advertising data, a content server that provides content, an SNS server that provides a social network service (SNS), a service server provided by a manufacturer, a multichannel video programming distributor (MVPD) that provides video on demand (VoD) or a streaming service, a service server that provides a pay service, or the like may be included as a server.

In an embodiment of the present disclosure, the device 100 means a server according to context, but may mean a fixed device or a mobile device, or may be used in an all-inclusive sense unless specified otherwise.

The processor 110 may generally control the overall operation of the device 100 in addition to an operation related to an application program. The processor 110 may provide or process appropriate information or functions by processing signals, data, or information that is input or output through the components of the device 100 or driving an application program stored in the storage 120.

The processor 110 may control at least some of the components of the device 100 to drive an application program stored in the storage 120. Furthermore, the processor 110 may operate by combining at least two or more of the components included in the device 100 to drive the application program.

The processor 110 may include one or more cores, and may be any of a variety of commercial processors. For example, the processor 110 may include a central processing unit (CPU), general purpose graphics processing unit (GPUGP), and tensor processing unit (TPU) of the device, but the present disclosure is not limited thereto.

The processor 110 of the present disclosure may be configured as a dual processor or other multiprocessor architecture, but the present disclosure is not limited thereto.

The processor 110 may identify the mild cognitive impairment using the mild cognitive impairment identification model according to some embodiments of the present disclosure by reading a computer program stored in the storage 120.

The storage 120 may store data supporting various functions of the device 100. The storage 120 may store a plurality of application programs (or applications) driven in the device 100, and data, commands, and at least one program command for the operation of the device 100. At least some of these application programs may be downloaded from an external server through wireless communication. In addition, at least some of these application programs may exist in the device 100 from the time of shipment for basic functions of the device 100. The application program may be stored in the storage 120, installed in the device 100, and driven by the processor 110 to perform the operation (or function) of the device 100.

The storage 120 may store any type of information generated or determined by the processor 110 and any type of information received through the communication unit 130.

The storage 120 may include at least one type of storage medium of a flash memory type, a hard disk type, a solid state disk (SSD) type, a silicon disk drive (SDD) type, a multimedia card micro type, a card-type memory (e.g., SD memory and XD memory), random access memory (RAM), static random access memory (SRAM), read-only memory (ROM), electrically erasable programmable read-only memory (EEPROM), programmable read-only memory (PROM), magnetic memory, a magnetic disk, and an optical disk. The device 100 may be operated in relation to a web storage that performs a storage function of the storage 120 on the Internet.

The communication unit 130 may include one or more modules that enable wire/wireless communication between the device 100 and a wire/wireless communication system, between the device 100 and another device, or between the device 100 and an external server. In addition, the communication unit 130 may include one or more modules that connect the device 100 to one or more networks.

The communication unit 130 refers to a module for wired/wireless Internet connection, and may be built-in or external to the device 100. The communication unit 130 may be configured to transmit and receive wire/wireless signals.

The communication unit 130 may transmit/receive a radio signal with at least one of a base station, an external terminal, and a server on a mobile communication network constructed according to technical standards or communication methods for mobile communication (e.g., Global System for Mobile communication (GSM), Code Division Multi Access (CDMA), Code Division Multi Access 2000 (CDMA2000), Enhanced Voice-Data Optimized or Enhanced Voice-Data Only (EV-DO), Wideband CDMA (WCDMA), High Speed Downlink Packet Access (HSDPA), High Speed Uplink Packet Access (HSUPA), Long Term Evolution (LTE), Long Term Evolution-Advanced (LTE-A), etc.).

Examples of wireless Internet technology include Wireless LAN (WLAN), Wireless-Fidelity (Wi-Fi), Wireless Fidelity (Wi-Fi) Direct, Digital Living Network Alliance (DLNA), Wireless Broadband (WiBro), World Interoperability for Microwave Access (WiMAX), High Speed Downlink Packet Access (HSDPA), High Speed Uplink Packet Access (HSUPA), Long Term Evolution (LTE), and Long Term Evolution-Advanced (LTE-A). However, in a range including Internet technologies not listed above, the communication unit 130 may transmit/receive data according to at least one wireless Internet technology.

In addition, the communication unit 130 may be configured to transmit and receive signals through short range communication. The communication unit 130 may perform short range communication using at least one of Bluetooth™, Radio Frequency Identification (RFID), Infrared Data Association (IrDA), Ultra-Wideband (UWB), ZigBee, Near Field Communication (NFC), Wireless-Fidelity (Wi-Fi), Wi-Fi Direct and Wireless Universal Serial Bus (Wireless USB) technology. The communication unit 130 may support wireless communication through short range communication networks (wireless area networks). The short range communication networks may be wireless personal area networks.

The device 100 according to some embodiments of the present disclosure may be connected to the user terminal 200 and the wire/wireless network 300 through the communication unit 130.

In an embodiment of the present disclosure, the user terminal 200 may be paired with or connected to the device 100, in which the mild cognitive impairment identification model is stored, over the wire/wireless network 300, thereby transmitting/receiving and displaying predetermined data.

The user terminal 200 described in the present disclosure may include any device that transmits, receives, and displays at least one of data, content, service, and application. In addition, the user terminal 200 may be a terminal of a user who wants to check mild cognitive impairment, but the present disclosure is not limited thereto.

In an embodiment of the present disclosure, the user terminal 200 may include, for example, a mobile device such as a mobile phone, a smart phone, a tablet PC, or an ultrabook, but the present disclosure is not limited thereto. The user terminal 200 may include a standing device such as a Personal Computer (PC), a microprocessor, a mainframe computer, a digital processor, or a device controller.

The user terminal 200 includes a processor 210, storage 220, a communication unit 230, an image acquisition unit 240, a display unit 250 and a sound output unit 260 The components illustrated in FIG. 1 are not essential in implementing the user terminal 200, and thus, the user terminal 200 described in the present disclosure may have more or fewer components than those listed above.

Each component of the user terminal 200 of the present disclosure may be integrated, added, or omitted according to the specifications of the user terminal 200 that is actually implemented. That is, as needed, two or more components may be combined into one component, or one component may be subdivided into two or more components. In addition, the function performed in each block is for describing an embodiment of the present disclosure, and the specific operation or device does not limit the scope of the present disclosure.

The processor 210, storage 220, and communication unit 230 of the user terminal 200 are the same components as the processor 110, storage 120, and communication unit 130 of the device 100, and thus redundant descriptions thereof will be omitted, and differences between them are chiefly described below.

In the present disclosure, the processor 210 of the user terminal 200 may control the display unit 250 to display the first object on the first region of the screen when performing a task to acquire digital biomarker data for identifying the presence or absence of mild cognitive impairment. Also, the processor 210 may control the display unit 250 to display a second object different from the first object on a second region different from the first region after the first object is displayed for a preset time.

Meanwhile, since high processing speed and computational power are required to perform an operation using the mild cognitive impairment identification model, the mild cognitive impairment identification model may be stored only in the storage 120 of the device 100 and may not be stored in the storage 220 of the user terminal 200, but the present disclosure is not limited thereto.

The image acquisition unit 240 may include one or a plurality of cameras. That is, the user terminal 200 may be a device including one or plural cameras provided on at least one of a front part and rear part thereof.

The image acquisition unit 240 may process an image frame, such as a still image or a moving image, acquired by an image sensor. The processed image frame may be displayed on the display unit 250 or stored in the storage 220. The image acquisition unit 240 provided in the user terminal 200 may match a plurality of cameras to form a matrix structure. A plurality of image information having various angles or focuses may be input to the user terminal 200 through the cameras forming the matrix structure as described above.

The image acquisition unit 240 of the present disclosure may include a plurality of lenses arranged along at least one line. The plurality of lenses may be arranged in a matrix form. The plural lenses may be arranged in a matrix form. Such cameras may be called an array camera. When the image acquisition unit 240 is configured as an array camera, images may be captured in various ways using the plural lenses, and images of better quality may be acquired.

According to some embodiments of the present disclosure, the image acquisition unit 240 may acquire an image including the user's eyes in conjunction with performing a task of acquiring digital biomarker data for identifying a user's mild cognitive impairment and a preliminary task to enable the user to check the task before performing the task.

Specifically, when the task or the preliminary task is performed, the image acquisition unit 240 may acquire an image including the user's eyes of the user terminal in conjunction with the display of the first object or the second object on the screen of the user terminal.

The display unit 250 may display (output) information processed by the user terminal 200. For example, the display unit 250 may display execution screen information of an application program driven in the user terminal 200, or user interface (UI) and graphic user interface (GUI) information according to the execution screen information.

The display unit 250 may include at least one of a liquid crystal display (LCD), a thin film transistor liquid crystal display (TFT LCD), an organic light-emitting diode (OLED), a flexible display, a 3D display, and an e-ink display, but the present disclosure is not limited thereto.

According to some embodiments of the present disclosure, the display unit 250 may display a first object (eg, a cross-shaped object) on the first region of the screen when performing a task or preliminary task. Also, the display unit 250 may display the second object instead of the first object on the second region of the screen when a predetermined time elapses after the first object is displayed.

The sound output unit 260 may output audio data (or sound data, etc.) received from the communication unit 230 or stored in the storage 220. The sound output unit 260 may also output a sound signal related to a function performed by the user terminal 200.

The sound output unit 260 may include a receiver, a speaker, or a buzzer. That is, the sound output unit 260 may be implemented as a receiver or may be implemented in the form of a loudspeaker, but the present disclosure is not limited thereto.

According to some embodiments of the present disclosure, the sound output unit 260 may output a preset sound (e.g., a voice describing what the user needs to do through a task or preliminary task) in conjunction with performing a task or preliminary task. However, it is not limited thereto.

According to some embodiments of the present disclosure, the processor 110 of the device 100 may acquire digital biomarker data using eye gaze information secured using an image including the user's eyes acquired through the image acquisition unit 240 of the user terminal 200. Here, the digital biomarker data may be acquired through a task for acquiring digital biomarker data.

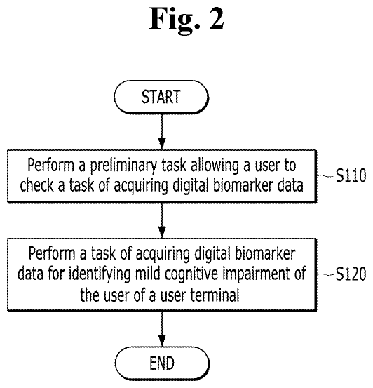

Meanwhile, in the present disclosure, before performing the task of acquiring digital biomarker data, a preliminary task for allowing the user to check the task may be performed. This will be described in more detail with reference to FIGS. 2 to 7.

FIG. 2 is a flowchart illustrating an example of a method of acquiring digital biomarker data according to some embodiments of the present disclosure. In describing FIG. 2, the contents overlapping with those described above in relation to FIG. 1 are not described again, and differences therebetween are mainly described below.

Referring to FIG. 2, the processor 110 of the device 100 may perform a preliminary task allowing the user to check a task of acquiring digital biomarker data (S110).

In the present disclosure, a preliminary task may include a first preliminary task causing a first object to be displayed on a first region of a screen displayed on the user terminal, a second preliminary task causing a second object to be displayed on a second region different from the first region on the screen of the user terminal instead of the first object, a third preliminary task of determining whether the user is gazing at a third region opposite to the second region based on the gaze information in conjunction with performing the second preliminary task, and a fourth preliminary task causing different information to be displayed on the screen of the user terminal based on whether the user is gazing at the third region.

After performing the preliminary task in step S110, the processor 110 of the device 100 may perform a task of acquiring digital biomarker data for identifying mild cognitive impairment of the user of the user terminal (S120).

In the present disclosure, a task may include a first task causing the first object to be displayed on the first region of the screen displayed on the user terminal after the fourth preliminary task is performed, a second task causing the second object to be displayed on the second region instead of the first object on the screen of the user terminal, and a third task of acquiring the digital biomarker data for identifying mild cognitive impairment of the user based on first gaze information acquired while performing the first task and the second task a preset number of times.

In the present disclosure, the first preliminary task and the second preliminary task included in the preliminary task may be the same as the first task and the second task included in the task. Accordingly, the user of the user terminal can check the contents of the corresponding task in advance before the task for acquiring digital biomarker data is performed. In addition, the user can smoothly perform a test (action performed by the user through the task) for identifying mild cognitive impairment with a high level of understanding of the contents of the task. In this case, it was experimentally confirmed that the accuracy of identifying mild cognitive impairment is improved.

Meanwhile, according to some embodiments of the present disclosure, the user's gaze information may be acquired in association with the processor 110 of the device 100 performing tasks and preliminary tasks. Here, the gaze information may mean information about the direction of the eyes indicating where the user is gazing.

As an example, the gaze information may be generated by the device 100 receiving an image including the user's eyes from the user terminal 200 and then analyzing the image by the processor 110 of the device 100. That is, the processor 110 of the device 100 may acquire gaze information by analyzing the corresponding image after the device 100 receives the image including the user's eyes acquired through the image acquisition unit 240 of the user terminal 200.

As another example, gaze information is information generated by the processor 210 of the user terminal 200 analyzing an image including the user's eyes, and may be information received by the device 100 from the user terminal. That is, the processor 210 of the user terminal 200 analyzes the image including the user's eyes acquired through the image acquisition unit 240 of the user terminal 200 to generate gaze information, the processor 110 of the device 100 may receive the corresponding gaze information through the communication unit 130 and the processor 110 of the device 100 may acquire the gaze information.

The above examples are only examples and the present disclosure is not limited to the above examples.

According to some embodiments of the present disclosure, the gaze information acquired in the preliminary task may not be used when identifying the presence or absence of mild cognitive impairment through a mild cognitive impairment identification model. However, it is not limited thereto, and in order to increase the accuracy of mild cognitive impairment identification of the mild cognitive impairment identification model, gaze information acquired in the preliminary task may also be input to the mild cognitive impairment identification model.

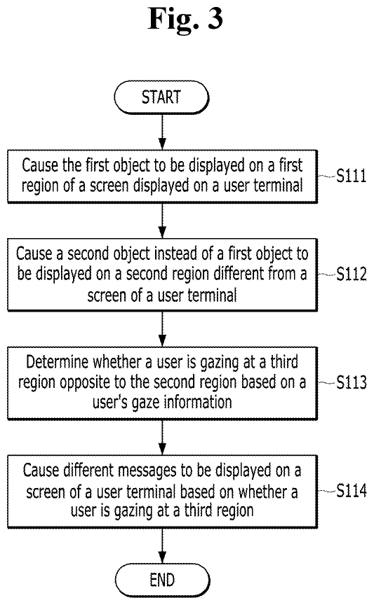

According to some embodiments of the present disclosure, before performing a task for acquiring digital biomarker data, a preliminary task may be performed first to increase the user's understanding of the task to be performed. This will be described in more detail with reference to FIGS. 3 to 5.

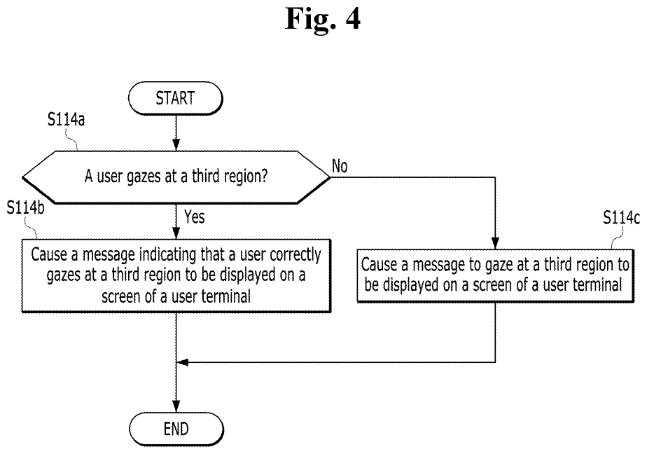

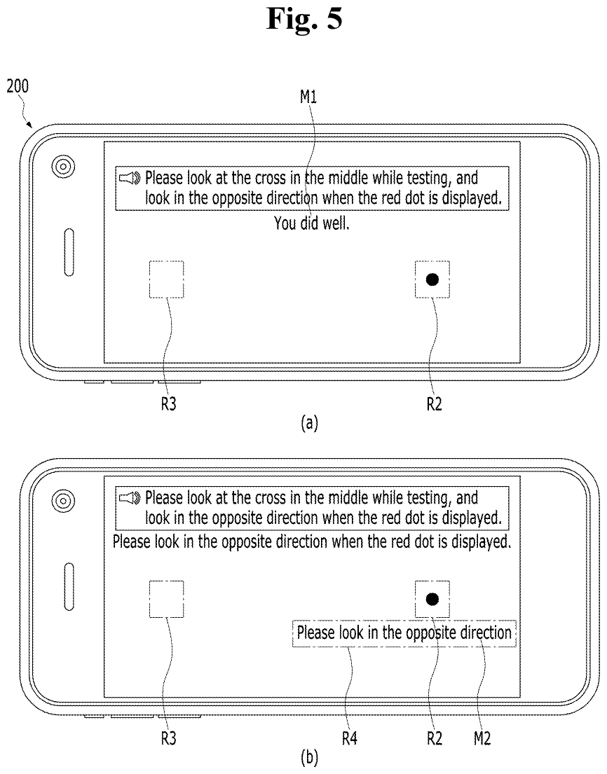

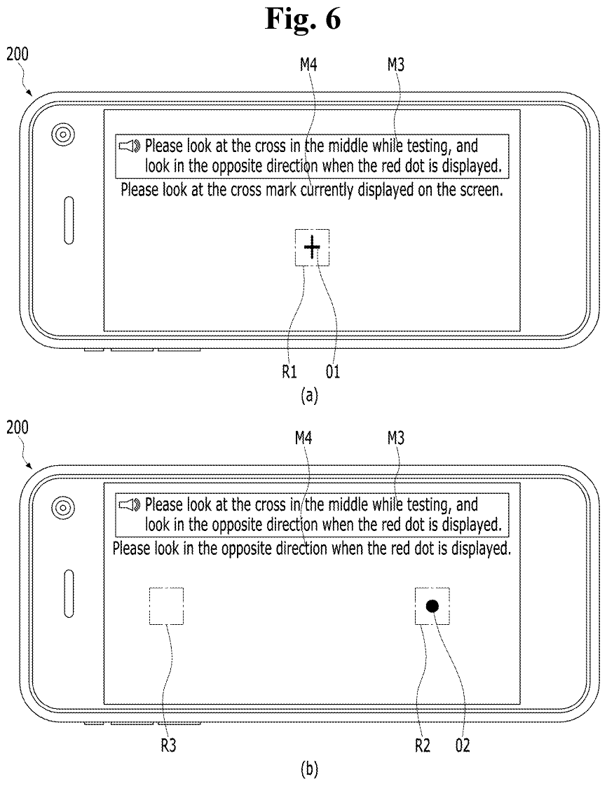

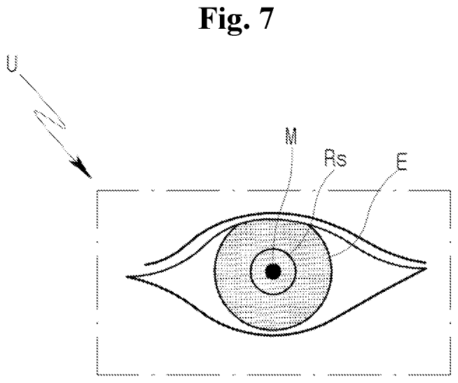

FIG. 3 is a flowchart illustrating an example of a method of performing a preliminary task according to some embodiments of the present disclosure. FIG. 4 is a flowchart illustrating an example of a method of outputting another message when performing a preliminary task according to some embodiments of the present disclosure. FIG. 5 is a flowchart illustrating an example of a message displayed when performing a preliminary task according to some embodiments of the present disclosure. FIG. 6 is a diagram for explaining an example of a screen displayed on a user terminal when a preliminary task or a task is performed according to some embodiments of the present disclosure. FIG. 7 is a diagram for explaining an example of a method of acquiring gaze information according to some embodiments of the present disclosure. In describing FIGS. 3-7, the contents overlapping with those described above in relation to FIGS. 1-2 are not described again, and differences therebetween are mainly described below.

Referring to FIG. 3, the processor 110 of the device 100 may perform a first preliminary task causing the first object to be displayed on the first region of the screen displayed on the user terminal (S111).

For example, the processor 110 of the device 100 may generate a screen including a first object on the first region and transmit it to the user terminal 200. In this case, the user terminal 200 may display a screen including the first object on the first region.

As another example, a screen in which the first object is included in the first region may be stored in the storage 220 of the user terminal 200. The display unit 250 may be controlled to display the screen on the user terminal 200 when the processor 210 of the user terminal 200 receives a signal from the device 100 to display the screen stored in the storage 220 through the communication unit 230.

As another example, the image of the first object may be stored in the storage 220 of the user terminal 200. In this case, the processor 210 of the user terminal 200 may generate and display a screen including the first object on the first region when the processor 110 of the device 100 transmits a signal to display the screen including the first object to the user terminal 200 through the communication unit 130.

However, since the above examples are only examples, the present disclosure is not limited to the above examples.

Referring to FIG. 6 (a), the screen displayed on the user terminal 200 may include the first object O1 in the first region R1.

The first object O1 may be an object that induces the user's gaze to come to the center of the displayed screen. For example, the first object O1 may be an object having a cross shape. However, the first object O1 is not limited to the above example and may have various forms or shapes.

The first region R1 may be an region located at the very center of the screen. Accordingly, the user's gaze gazing at the first object O1 may come to the center of the screen. However, the present disclosure is not limited thereto.

Referring back to FIG. 3, the processor 110 of the device 100 may perform a second preliminary task causing a second object to be displayed on a second region different from the first region on the screen of the user terminal 200 instead of the first object (S112).

Specifically, the processor 110 of the device 100 may cause a second object to be displayed instead of the first object on a second region different from the first region when recognizing that a preset time (eg, 3 to 5 seconds) has elapsed while the first object is displayed on the screen of the user terminal 200.

For example, the processor 110 of the device 100 may generate a screen including a second object on the second region and transmit it to the user terminal 200 when it is recognized that the first object is displayed on the first region for a preset time. In this case, the user terminal 200 may display a screen including the second object on the second region.

As another example, a screen in which the second object is included in the second region may be stored in the storage 220 of the user terminal 200. The processor 210 of the user terminal 200 may control the display unit 250 to display the screen on the user terminal 200 when the device 100 receives a signal to display the screen stored in the storage 220 through the communication unit 230. Here, the signal may be transmitted from the device 100 to the user terminal 200 when it is recognized that the first object is displayed on the first region for a preset time.

As another example, the image of the second object may be stored in the storage unit 220 of the user terminal 200. In this case, the processor 210 of the user terminal 200 may generate and display a screen including the second object on the second region, when the processor 110 of the device 100 transmits a signal to display the screen including the second object to the user terminal 200 through the communication unit 130. Here, the signal may be transmitted from the device 100 to the user terminal 200 when it is recognized that the first object is displayed on the first region for a preset time.

However, since the above examples are only examples, the present disclosure is not limited to the above examples.

On the other hand, referring to FIG. 6 (b), the second object O2 may be an object having a preset shape (eg, a circular shape with a diameter of 0.2 cm, etc.) and a preset color (eg, red). Also, the second region R2 may be positioned to the right or left of the first region R1 positioned at the center. Here, the position of the second region R2 may be randomly selected from the right side or the left side of the first region R1. However, it is not limited thereto.

In the present disclosure, the screen on which the second object O2 is displayed may be displayed for 2000 ms. However, it is not limited thereto.

Meanwhile, according to some embodiments of the present disclosure, a message M3 indicating what the user should do when performing the test progresses and a message M4 indicating what the user should do through the currently displayed screen may be displayed on the screen displayed on the user terminal 200.

For example, referring to FIG. 6 (a) and FIG. 6 (b), the message M3 indicating what the user should do when performing the test may include content such as “Please look at the cross in the middle while testing, and look in the opposite direction when the red dot is displayed.”. Referring to FIG. 6 (a), the message M4 indicating what the user should do through the currently displayed screen may include content such as “Please look at the first object O1 currently displayed on the screen.”. Referring to FIG. 6 (b), the message M4 indicating what the user should do through the currently displayed screen may include content such as “Please look in the opposite direction when the red dot is displayed.”.

In addition, the user terminal 200 may output a sound (e.g., a voice explaining the content contained in the message M4) related to the message M4 through the sound output unit 260 in conjunction with displaying the message M4 on the screen through the display unit 250. In this way, when a sound is output together with the message M4 related to the currently displayed screen so that the user recognizes the task to be performed, the user can clearly understand what the task to be performed is. Therefore, the possibility of performing an incorrect operation by simple mistake may be reduced.

Referring back to FIG. 3, the processor 110 of the device 100 may determine whether the user is gazing at a third region opposite to the second region based on the user's gaze information acquired in the second preliminary task (S113).

In the present disclosure, gaze information may be acquired by analyzing an image including the user's eyes acquired while performing the second preliminary task. Here, the gaze information may be information representing the movement of the user's eyes.

For example, the processor 110 of the device 100 may receive an image including the user's eyes from the user terminal 200 through the communication unit 130 while the second preliminary task is being performed. Also, when the image is received, the processor 110 may generate gaze information by analyzing the image.

As another example, the processor 210 of the user terminal 200 may acquire an image including the user's eyes while the second preliminary task is being performed. The processor 210 may acquire gaze information by analyzing the acquired image. Also, the processor 210 may control the communication unit 230 to transmit the acquired gaze information to the device 100. In this case, the processor 110 of the device 100 may receive gaze information from the user terminal 200 through the communication unit 130.

A method of acquiring gaze information in the present disclosure will be described in more detail with reference to FIG. 7. For convenience of description, a method for acquiring gaze information by the processor 110 of the device 100 will be described below, but the processor 210 of the user terminal 200 may acquire gaze information in the same manner.

Referring to FIG. 7, the processor 110 of the device 100 may determine the location of the pupil E of the user U in each of a plurality of frames by using only a B value among respective RGB values of the plural frames included in the acquired image. That is, the processor 110 may recognize that a region having a B value exceeding a preset threshold value in each of the plural frames is a region where the pupil E is located. In addition, the processor 110 may acquire gaze information based on the position of the center point of the region where the pupil E is located. However, the present invention is not limited thereto, and the above-described method of confirming the position of the pupil E may be performed by the processor 210 of the user terminal 200.

According to some embodiments of the present disclosure, the processor 110 of the device 100 may acquire gaze information based on the coordinate value of the pupil E of the user. Here, the coordinate value of the pupil E may be the coordinate value of the central point M of the pupil E or may be the coordinate values of the edge of the pupil E. However, the present invention is not limited thereto.

According to some embodiments of the present disclosure, the processor 110 may distinguish the pupil E from the background in the acquired image. In addition, the processor 110 may undergo a binarization process of changing a part corresponding to the position of the pupil E to black and a part corresponding to the background to white. In addition, the processor 110 may apply a flood fill to remove noise after the binarization process. Here, the flood fill may refer to an operation of replacing white pixels surrounded by black pixels with black pixels and replacing black pixels surrounded with white pixels. Next, the processor 110 may acquire gaze information by checking the location of the central point of the pupil using the acquired image.

Referring to FIG. 6 (b), the processor 110 may check whether the location of the central point M of the user's pupil E is located within the preset area Rs in order to determine whether the user is gazing at the third region R3.

Referring back to FIG. 7, the preset area Rs may mean an area where the center point M of the user's pupil E is located when the user gazes at the third region (R3 in FIG. 6(b)).

For example, as shown in FIG. 6 (b), when the third region R3 is located on the left side of the first region R1 in FIG. 6 (a), the preset area Rs may also be located on the left side corresponding to this, and when the third region R3 is located on the right side of the first region R1, the preset area Rs may also be located on the right side correspondingly. According to some embodiments of the present disclosure, the size of the preset area Rs may be determined according to the size of the pupil E. If the size of the preset area Rs varies according to the size of the pupil, the accuracy of determining whether the user gazes at the third region R3 may be improved. This is because the size of the user's pupil E may be different for each user.

Specifically, when an image including the eyes of the user U is acquired, the processor 110 may recognize the size of the pupil E by analyzing the image. Also, the processor 110 may determine the size of the preset region Rs based on the size of the pupil E. The size of the preset region Rs may be proportional to the size of the pupil E. Also, the size of the preset area Rs may be smaller than the size of the pupil E.

Referring back to FIG. 3, the processor 210 of the user terminal 200 may acquire an image including the user's eyes by activating the image acquisition unit 240 while the screen including the second object O2 is displayed. That is, while the second preliminary task is being performed in step S112, the processor 210 may check whether the user's eyes are included in the image. The processor 210 may control the display unit 250 to display a screen on which the second object O2 is located on the second region R2 while continuously acquiring an image if the processor 210 recognizes that the user's eyes are not included in the image. However, it is not limited thereto.

According to some embodiments of the present disclosure, the processor 210 of the user terminal 200 may control the display unit 250 to continuously display a message instructing the user to gaze at the second object when it is recognized that the user's eyes are not included in the image acquired through the image acquisition unit 240 while the screen including the second object is displayed. In addition, the processor 210 may control the sound output unit 260 to output a voice prompting the user to gaze at the second object in conjunction with displaying the message. However, the present disclosure is not limited to the above contents.

Referring back to FIG. 3, the processor 110 may cause different messages to be displayed on the screen of the user terminal 200 based on whether the user is gazing at the third region (S114).

Specifically, referring to FIG. 4, the processor 110 may first check whether the user gazes at the third region based on gaze information (S114a).

For example, the processor 110 may determine whether the central point of the user's pupil exists in a preset area (area corresponding to the third region (Rs in FIG. 7) based on gaze information acquired by analyzing an image including the user's eyes. Further, the processor 110 may recognize that the user is gazing at the third region when recognizing that the central point exists in the preset area.

As another example, the processor 110 may determine whether the coordinate value of the user's pupil is within a specific range based on gaze information acquired by analyzing an image including the user's eyes. Further, the processor 110 may recognize that the user is gazing at the third region when recognizing that the coordinate value of the user's pupil exists within the specific range.

The above examples are examples and the present disclosure is not limited to the above examples.

Meanwhile, when the processor 110 recognizes that the user gazes at the third region (S114a, Yes), the processor 110 may cause a message indicating that the user correctly gazes at the third region to be displayed on the screen of the user terminal 200.

For example, referring to FIG. 5 (a), when it is recognized that the user gazes at the third region R3 opposite to the second region R2, the processor 110 may cause a message M1 saying “You did well” to be displayed on the screen of the user terminal 200.

Meanwhile, referring to FIG. 4 again, when it is recognized that the user does not gaze at the third region (S114a, No), the processor 110 may cause a message to gaze at the third region to be displayed on the screen of the user terminal 200 (S114c).

For example, referring to FIG. 5 (b), when it is recognized that the user does not gaze at the third region R3 opposite to the second region R2, the processor 110 may cause a message M2 saying “Please look in the opposite direction” to be displayed on the screen of the user terminal 200. Here, the message M2 requesting the user to gaze at the third region may be displayed on the peripheral region R4 of the second region.

In the present disclosure, the peripheral region R4 may be an region disposed within a preset distance in any one of the top/bottom/left/right directions of the second region R2. The size of the peripheral region R4 may correspond to the size of the message M2 requesting the user to gaze at the third region. However, the present disclosure is not limited to the above.

Users generally have a tendency to gaze at the second object when the second object is displayed in the second region. Therefore, when a message prompting the user to gaze at the third region is displayed on the peripheral region R4 of the second region if the user does not gaze at the third region, the possibility that the user gazes at the third region increases according to the content of the message. That is, through the preliminary task, the user can clearly recognize what the user has to perform.

Meanwhile, according to some embodiments of the present disclosure, the first message displayed when the user recognizes that he or she is gazing at the third region and the second message displayed when recognizing that the user does not gaze at the third region may be displayed in different regions.

It is preferable that the message displayed when the user properly gazes at the third region is displayed at a position where the user is not obstructed from proceeding with the test. However, the message displayed when the user does not properly gaze at the third region should be displayed at a location where the user mainly gazes incorrectly. This is because the user can correctly recognize the test content only when the corresponding message is displayed at the corresponding position. Accordingly, it may be most suitable for the second message to be displayed on the peripheral region R4 when it is recognized that the user is not properly gazing at the third region.

According to some embodiments of the present disclosure, the processor 110 may control the first preliminary task S111, the second preliminary task S112, the third preliminary task S113, and the fourth preliminary task S114 shown in FIG. 3 to be repeated as many as a preset first number of times.

That is, the processor 210 of the user terminal 200 may repeat an operation of acquiring an image including the user's eye through the image acquisition unit 240 a preset number of times while controlling the display unit 250 to display the second object instead of the first object on the second region after displaying the first object on the first region of the screen.

In this case, when it is recognized that the user's gaze is not gazing at the third region based on the user's gaze information acquired by analyzing the image acquired through the image acquisition unit 240, the processor 210 of the user terminal 200 may control the display unit 250 to display a message to gaze at the third region on the screen. And, when it is recognized that the user's gaze is gazing at the third region based on the user's gaze information acquired by analyzing the image acquired through the image acquisition unit 240, the processor 210 of the user terminal 200 may control the display unit 250 to display a message indicating that the user is properly gazing at the third region on the screen.

According to some embodiments of the present disclosure, the processor 110 of the device 100 may perform a task of acquiring digital biomarker data for identifying mild cognitive impairment when the preliminary task is completed a preset number of times. In this case, the processor 110 may cause a screen including a selection button for selecting whether to perform the preliminary task once again or immediately perform the task to be displayed on the screen of the user terminal 200. However, the present disclosure is not limited thereto.

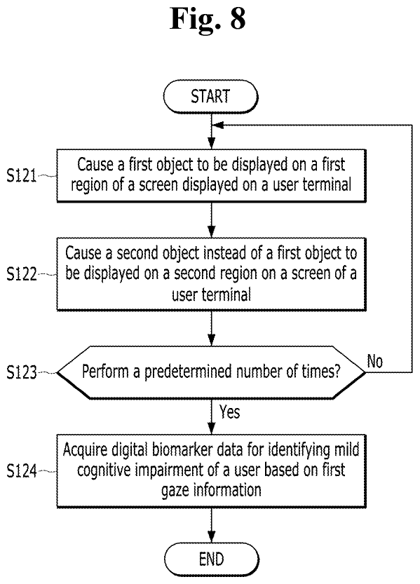

Meanwhile, the task of acquiring digital biomarker data for identifying mild cognitive impairment may be performed through a process similar to that of the preliminary task. This will be described later with reference to FIG. 8.

FIG. 8 is a flowchart illustrating a process of performing a task of acquiring digital biomarker data according to some embodiments of the present disclosure. In describing FIGS. 8, the contents overlapping with those described above in relation to FIGS. 1-7 are not described again, and differences therebetween are mainly described below.

Referring to FIG. 8, the processor 110 of the device 100 may perform a first task causing a first object to be displayed on a first region of a screen displayed on the user terminal 200 (S121). Here, since the first task is the same as the first preliminary task S111 described above with reference to FIG. 3, a detailed description thereof will be omitted.

Meanwhile, when it is recognized that the first object has been displayed on the first region for a preset time, the processor 110 of the device 100 may perform a second task causing a second object instead of the first object to be displayed on the second region on the screen of the user terminal (S122). Here, since the second task is the same as the second preliminary task S112 described above with reference to FIG. 3, a detailed description thereof will be omitted.

As described above, since the user knows what tasks to be performed through the first preliminary task and the second preliminary task before performing the first task and the second task, it can solve the problem that the user does not understand the contents of the test and does not perform the test properly. As in the present disclosure, it was experimentally confirmed that the accuracy of identifying mild cognitive impairment is improved when the preliminary task is performed first and then the task is performed.

Meanwhile, the processor 110 of the device 100 may check whether the first task and the second task have been performed a predetermined number of times (S123). Here, the preset number of times may be greater than the preset first number of repetitions of the preliminary task.

In the present disclosure, when it is recognized that the first task (S121) and the second task (S122) have not been performed a preset number of times (S123, No), the processor 110 of the device 100 may repeatedly perform the first task S121 and the second task S122 so that the first task S121 and the second task S122 are performed a predetermined number of times.

Meanwhile, when it is recognized that the first task (S121) and the second task (S122) have been performed a predetermined number of times (S123, Yes), the processor 110 of the device 100 may acquire digital biomarker data for identifying mild cognitive impairment of the user based on the first gaze information (S124).

In the present disclosure, the digital biomarker data may comprise at least one of information on a number of times the user gazed at the third region, information on a number of times the user gazed at a region other than the third region, information on whether the user's gaze continues to stare at a specific point for the preset time, information on a time elapsed for the user's gaze to move while performing the second task (information about how long the user's response was delayed), information on movement speed of the user's gaze and information on whether the user's gaze is accurately staring at the third region. However, it is not limited thereto, and the digital biomarker data may include less or more information than the above-described information. In addition, in order to improve the accuracy of mild cognitive impairment identification of the mild cognitive impairment identification model, digital biomarker data may include all of the above-described information.

The above-described digital biomarker data of the present disclosure may be a digital biomarker having a high correlation coefficient with identification of mild cognitive impairment among various types of digital biomarkers. Accordingly, when determining whether mild cognitive impairment exists using the digital biomarker data described above, the accuracy of identifying mild cognitive impairment may be improved.

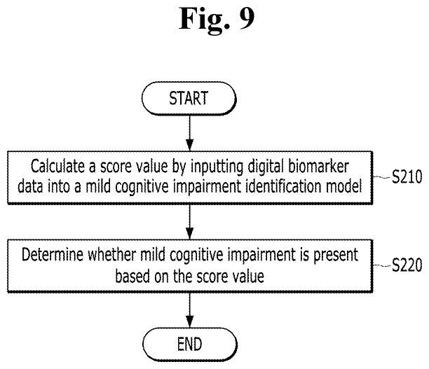

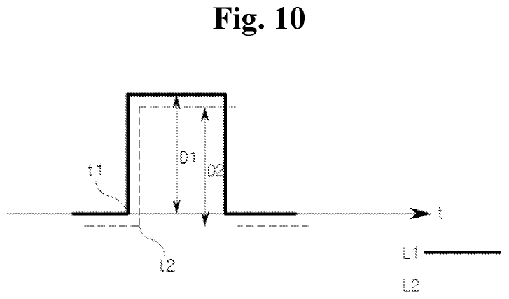

FIG. 9 is a flowchart illustrating an example of a method of determining whether a user has mild cognitive impairment using digital biomarker data according to some embodiments of the present disclosure. FIG. 10 is a diagram for explaining an example of information included in digital biomarker data according to some embodiments of the present disclosure. In describing FIGS. 9 and 10, the contents overlapping with those described above in relation to FIGS. 1-8 are not described again, and differences therebetween are mainly described below.

Referring to FIG. 9, the processor 110 of the device 100 may calculate a score value by inputting digital biomarker data into a mild cognitive impairment identification model (S210).

In the present disclosure, the digital biomarker data may comprise at least one of information on a number of times the user gazed at the third region, information on a number of times the user gazed at a region other than the third region, information on whether the user's gaze continues to stare at a specific point for the preset time, information on a time elapsed for the user's gaze to move while performing the second task (information about how long the user's response was delayed), information on movement speed of the user's gaze and information on whether the user's gaze is accurately staring at the third region. However, it is not limited thereto, and the digital biomarker data may include less or more information than the above-described information. In addition, in order to improve the accuracy of mild cognitive impairment identification of the mild cognitive impairment identification model, digital biomarker data may include all of the above-described information.

In the present disclosure, the processor 110 may calculate the number of times the user gazes at the third area and the number of times the user gazes at areas other than the third area among the predetermined number of times when the first task and the second task are performed a preset number of times.

In the present disclosure, information on whether the user's gaze continues to gaze at a specific point for a preset time period may be checked based on whether the user's pupil moves at a specific location at a specific time point.

For example, information on whether the user's gaze continues to gaze at a specific point for a preset time period may be determined based on whether the user's pupil moves at a specific location while the first object is being displayed. Here, the specific position may be a position at which the pupil gazes at the first object, and it may be determined that the pupil has moved when the coordinate value of the pupil deviates from a preset threshold range.

As another example, information about whether the user's gaze continues to gaze at a specific point for a preset time may be determined based on whether or not the pupil of the user has stopped moving for a predetermined time at the last stopped position after moving the user's pupil. Here, the specific position may be a position at which the pupil gazes at the second object O2 of FIG. 6, and it may be determined that the pupil has moved when the coordinate value of the pupil deviates from a preset threshold range.

Accuracy of identifying mild cognitive impairment can be improved when information on whether the user's gaze continues to gaze at a specific point for a predetermined time is obtained and used as digital biomarker data for identifying mild cognitive impairment. This is because patients with mild cognitive impairment cannot gaze at one point for a long time.

Meanwhile, referring to FIG. 10, a method of acquiring information included in digital biomarker data for identifying mild cognitive impairment will be described in detail as follows.

In FIG. 10, the x-axis is an axis related to time, and the y-axis may be an axis related to a distance moved by the gaze or to a distance from the center of the screen to an object stared by a user. In addition, a first line L1 is a line representing a distance from the center of a screen to an object that a user should stare over time, and a second line L2 may be a line representing a distance by which the user's gaze moves over time.

In the present disclosure, a method of acquiring gaze information when the screen described with reference to FIG. 6 is displayed is described below.

In the present disclosure, the first time point t1 may be a time point when a screen including a second object is displayed, as shown in FIG. 6 (b).

A time point at which the user's gaze begins to move after the screen including the second object is displayed may be the second time point t2. Here, the second time point t2 may be a time point when the coordinates of the pupil start to move in a a stationary state. However, it is not limited thereto.

In the present disclosure, the delay time (the time elapsed for the user's gaze to move while performing the second task) of a user's response may mean the time elapsed from the first time point t1, when the screen including the second object is displayed, to the second time point t2, when the user's gaze moves.

Meanwhile, information on whether the user's gaze is accurately gazing at the third region can be checked using the distance D2 of the user's gaze movement and the distance D1 from the first region (R1 in FIG. 6 (a)) to the third region (R3 in FIG. 6 (b)). Here, the distance D2 by which the user's gaze has moved may be calculated using an initial coordinate value of the pupil (a coordinate value at the point where the pupil is located before the pupil moves) and a final coordinate value of the pupil (a coordinate value at a last point where the pupil stops after moving).

Specifically, information on whether the user's gaze is accurately gazing at the third region (R3 in FIG. 6 (b)) (i.e., information about whether the user is properly gazing at the object to be gazed at) may be checked using a value acquired by dividing the second distance D2 by the first distance D1. Here, as the value approaches 1, it may be considered that the user's gaze accurately stares at the point (the second object or the third object) related to the preset gaze task.

In the present disclosure, the movement speed of the user's gaze may be calculated by differentiating the position trajectory shown in FIG. 10 and reducing the velocity value. However, the present invention is not limited thereto, and the movement velocity may be calculated based on information on a distance where the user's eye moves from the center to a target point and information on a time taken when the user's eye moves from the center to the target point. The processor 110 may calculate the movement velocity of the user's gaze in various ways.

Referring again to FIG. 9, the mild cognitive impairment identification model in step S210 may mean an artificial intelligence model having a pre-learned neural network structure to calculate a score value when gaze information is input. And, the score value may mean a value capable of recognizing whether or not mild cognitive impairment exists according to the size of the value.

According to some embodiments of the present disclosure, a pre-learned mild cognitive impairment identification model may be stored in the storage 120 of the device 100.

The mild cognitive impairment identification model is learned by backpropagating the difference value (error) between the label data labeled in the data for learning and the predicted data output from the mild cognitive impairment identification model to update the weight of the mild cognitive impairment identification model.

In the present disclosure, data for learning may be digital biomarker data acquired by performing the above-described first and second tasks by a plurality of test users through their test devices.

In an embodiment of the present disclosure, the test users may include a user classified as a patient with mild cognitive impairment (MCI), a user classified as an Alzheimer's patient, a user classified as normal, but the present disclosure is not limited thereto.

In an embodiment of the present disclosure, the test device may refer to a device where various test users perform tests when securing input data for learning. In this case, the test device may be a mobile device, such as a mobile phone, a smart phone, a tablet PC, or an ultrabook, similarly to the user terminal 200 used for mild cognitive impairment identification, but the present disclosure is not limited thereto.

In an embodiment of the present disclosure, the label data may be a score value capable of recognizing whether a patient is normal, is an Alzheimer's patient, and a patient with mild cognitive impairment, but the present disclosure is not limited thereto.

A mild cognitive impairment identification model may be composed of a set of interconnected computational units, which may generally be referred to as nodes. These nodes may also be referred to as neurons. The neural network may be configured to include at least one node. Nodes (or neurons) constituting the neural network may be interconnected by one or more links.

In the mild cognitive impairment identification model, one or more nodes connected through a link may relatively form a relationship between an input node and an output node. The concepts of an input node and an output node are relative, and any node in an output node relationship with respect to one node may be in an input node relationship in a relationship with another node, and vice versa. As described above, an input node-to-output node relationship may be created around a link. One output node may be connected to one input node through a link, and vice versa.

In the relation between the input node and the output node connected through one link, a value of data of the output node may be determined based on data that is input to the input node. In this case, the link interconnecting the input node and the output node may have a weight. The weight may be variable, and may be changed by a user or an algorithm so that the neural network performs a desired function.

For example, when one or more input nodes are connected to one output node by each link, the output node may determine an output node value based on values that are input to input nodes connected to the output node and based on a weight set in a link corresponding to each input node.

As described above, in the mild cognitive impairment identification model, one or more nodes may be interconnected through one or more links to form an input node and output node relationship in the neural network. The characteristics of the mild cognitive impairment identification model may be determined according to the number of nodes and links in the mild cognitive impairment identification model, a correlation between nodes and links, and a weight value assigned to each of the links.

The mild cognitive impairment identification model may consist of a set of one or more nodes. A subset of nodes constituting the mild cognitive impairment identification model may constitute a layer. Some of the nodes constituting the mild cognitive impairment identification model may configure one layer based on distances from an initial input node. For example, a set of nodes having a distance of n from the initial input node may constitute n layers. The distance from the initial input node may be defined by the minimum number of links that should be traversed to reach the corresponding node from the initial input node. However, the definition of such a layer is arbitrary for the purpose of explanation, and the order of the layer in the mild cognitive impairment identification model may be defined in a different way from that described above. For example, a layer of nodes may be defined by a distance from a final output node.

The initial input node may refer to one or more nodes to which the input data for learning (i.e. gaze information) is directly input without going through a link in a relationship with other nodes among nodes in the mild cognitive impairment identification model. Alternatively, in a relationship between nodes based on a link in the mild cognitive impairment identification model, it may mean nodes that do not have other input nodes connected by a link. Similarly, the final output node may refer to one or more nodes that do not have an output node in relation to other nodes among nodes in the mild cognitive impairment identification model. In addition, a hidden node may refer to nodes constituting the mild cognitive impairment identification model other than the first input node and the last output node.

In the mild cognitive impairment identification model according to some embodiments of the present disclosure, the number of nodes in the input layer may be greater than the number of nodes in the output layer, and the neural network may have a form wherein the number of nodes decreases as it progresses from the input layer to the hidden layer. In addition, information on a number of times the user gazed at the third region, information on a number of times the user gazed at a region other than the third region, information on whether the user's gaze continues to stare at a specific point for the preset time, information on a time elapsed for the user's gaze to move while performing the second task, information on movement speed of the user's gaze and information on whether the user's gaze is accurately staring at the third region may be inputted in each node of the input layer. However, the present invention is not limited thereto.

According to some embodiments of the present disclosure, the mild cognitive impairment identification model may have a deep neural network structure.

A deep neural network (DNN) may refer to a neural network including a plurality of hidden layers in addition to an input layer and an output layer. The DNN may be used to identify the latent structures of data.

The DNN may include convolutional neural networks (CNNs), recurrent neural networks (RNNs), auto encoders, generative adversarial networks (GANs), and restricted Boltzmann machines (RBM), a deep belief network (DBN), a Q network, a U network, a Siamese network, and a generative adversarial network (GAN). These DNNs are only provided as examples, and the present disclosure is not limited thereto.

The mild cognitive impairment identification model of the present disclosure may be learned in a supervised learning manner, but the present disclosure is not limited thereto. The mild cognitive impairment identification model may be learned in at least one manner of unsupervised learning, semi supervised learning, or reinforcement learning.

Learning of the mild cognitive impairment identification model may be a process of applying knowledge for performing an operation of identifying the mild cognitive impairment by the mild cognitive impairment identification model to a neural network.

The mild cognitive impairment identification model can be trained in a way to minimize output errors. Learning of the mild cognitive impairment identification model is a process of repeatedly inputting the input data for learning into the mild cognitive impairment identification model, calculating errors of an output (score value predicted through the neural network) and target (score value used as label data) of the mild cognitive impairment identification model on the input data for learning, and updating the weight of each node of the mild cognitive impairment identification model by backpropagating the error of the mild cognitive impairment identification model from an output layer of the mild cognitive impairment identification model to an input layer in a direction of reducing the error.

A change amount of a connection weight of each node to be updated may be determined according to a learning rate. Calculation of the mild cognitive impairment identification model on the input data and backpropagation of errors may constitute a learning cycle (epoch). The learning rate may be differently applied depending on the number of repetitions of a learning cycle of the mild cognitive impairment identification model. For example, in an early stage of learning the mild cognitive impairment identification model, a high learning rate may be used to enable the mild cognitive impairment identification model to quickly acquire a certain level of performance, thereby increasing efficiency, and, in a late stage of learning the mild cognitive impairment identification model, accuracy may be increased by using a low learning rate.

In the learning of the mild cognitive impairment identification model, the input data for learning may be a subset of actual data (i.e., data to be processed using the learned mild cognitive impairment identification model), and thus, there may be a learning cycle wherein errors for the input data for learning decrease but errors for real data increase. Overfitting is a phenomenon wherein errors on actual data increase due to over-learning on input data for learning as described above.

Overfitting may act as a cause of increasing errors in a machine learning algorithm. To prevent such overfitting, methods, such as increasing the input data for learning, regularization, and dropout that deactivate some of nodes in a network during a learning process, and the utilization of a batch normalization layer, may be applied.

Meanwhile, when a score value is acquired through step S210, the processor 110 may determine whether mild cognitive impairment is present based on the score value (S220).

Specifically, the processor 110 may determine whether mild cognitive impairment is present based on whether the score value exceeds a preset threshold value.

For example, the processor 110 may determine that a user has mild cognitive impairment when recognizing that the score value output from the mild cognitive impairment identification model exceeds the preset threshold value.

As another example, the processor 110 may determine that a user does not have mild cognitive impairment when recognizing that the score value output from the mild cognitive impairment identification model is less than or equal to the preset threshold value.

The above-described embodiments are only provided as examples, and the present disclosure is not limited to the embodiments.

According to some embodiments of the present disclosure, the processor 110 of the device 100 may acquire user identification information before proceeding with a preliminary task and second task. Here, the user identification information may include user's age information, gender information, name, address information, etc. In addition, at least a portion of the user identification information may be used as input data for the mild cognitive impairment identification model together with the digital biomarker data. Specifically, age information and gender information may be used as input data for the mild cognitive impairment identification model together with gaze information. In this way, when at least a portion of the user identification information is used together with the digital biomarker data and is input to the mild cognitive impairment identification model to acquire a score value and identify whether or not the mild cognitive impairment is present, the accuracy of mild cognitive impairment identification may be further improved. In this case, the mild cognitive impairment identification model may be a model wherein learning is completed based on at least a part of user identification information, and digital biomarker data.

120 people in a normal cognitive group and 9 people in a cognitively impaired group conducted experiments in order to identify whether they had dementia by using their user terminals. Specifically, the device 100 determine the existence of mild cognitive impairment based on the score value generated by inputting the digital biomarker data acquired by performing the first task and the second task a preset number of times into the mild cognitive impairment identification model of the present disclosure. It was confirmed that the accuracy of the classification calculated through the above-described experiment was 80% or more.

According to at least one of the aforementioned several embodiments of the present disclosure, mild cognitive impairment may be accurately diagnosed in a way that a patient rarely feels rejection.

In an embodiment of the present disclosure, the configurations and methods of the aforementioned several embodiments of the device 100 are not limitedly applied, and all or parts of each of the embodiments may be selectively combined to allow various modifications.

Various embodiments described in the present disclosure may be implemented in a computer or similar device-readable recording medium using, for example, software, hardware, or a combination thereof.

According to hardware implementation, some embodiments described herein may be implemented using at least one of application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, and other electrical units for performing functions. In some cases, some embodiments described in the present disclosure may be implemented with at least one processor.

According to software implementation, some embodiments such as the procedures and functions described in the present disclosure may be implemented as separate software modules. Each of the software modules may perform one or more functions, tasks, and operations described in the present disclosure. A software code may be implemented as a software application written in a suitable programming language. In this case, the software code may be stored in the storage 120 and executed by at least one processor 110. That is, at least one program command may be stored in the storage 120, and the at least one program command may be executed by the at least one processor 110.

The method of identifying mild cognitive impairment by the at least one processor 110 of the device 100 using the mild cognitive impairment identification model according to some embodiments of the present disclosure may be implemented as code readable by the at least one processor in a recording medium readable by the at least one processor 110 provided in the device 100. The at least one processor-readable recording medium includes all types of recording devices in which data readable by the at least one processor 110 is stored. Examples of the at least one processor-readable recording medium includes read only memory (ROM), random access memory (RAM), CD-ROM, a magnetic tape, a floppy disk, and an optical data storage device.

Although the present disclosure has been described with reference to the accompanying drawings, this is only an embodiment and the present disclosure is not limited to a specific embodiment. Various contents that can be modified by those of ordinary skill in the art to which the present disclosure belongs also belong to the scope of rights according to the claims. In addition, such modifications should not be understood separately from the technical spirit of the present disclosure.

What is claimed is:

1. A method of identifying mild cognitive impairment by at least one processor of a device, the method comprising:
performing a task of acquiring digital biomarker data for identifying mild cognitive impairment of a user of a user terminal;
performing a preliminary task to allow the user to check the task before performing the task; and
acquiring gaze information of the user in conjunction with performing the task and the preliminary task;
wherein the gaze information is used when determining displayed information in the preliminary task, and used when identifying mild cognitive impairment of the user in the task.

2. The method according to claim 1, wherein the preliminary task includes:
a first preliminary task causing a first object to be displayed on a first region of a screen displayed on the user terminal;
a second preliminary task causing a second object to be displayed on a second region different from the first region on the screen of the user terminal instead of the first object;
a third preliminary task of determining whether the user is gazing at a third region opposite to the second region based on the gaze information in conjunction with performing the second preliminary task; and
a fourth preliminary task causing different information to be displayed on the screen of the user terminal based on whether the user is gazing at the third region.

3. The method according to claim 2, wherein the fourth preliminary task includes:
a first information output task causing first information that the user is gazing at the third region to be displayed on the screen of the user terminal if it is recognized that the user's gaze is gazing at the third region based on the gaze information; or
a second information output task causing second information prompting the user to gaze at the third region to be displayed on the screen of the user terminal when it is recognized that the gaze of the user does not gaze at the third region based on the gaze information.

4. The method according to claim 3, wherein the second information is displayed in a region different from a region in which the first information is displayed on the screen of the user terminal.

5. The method according to claim 3, wherein the second information is displayed on a peripheral region of the second region where the second object is displayed.

6. The method according to claim 2, wherein the task includes:
a first task causing the first object to be displayed on the first region of the screen displayed on the user terminal after the fourth preliminary task is performed;
a second task causing the second object to be displayed on the second region instead of the first object on the screen of the user terminal; and
a third task of acquiring the digital biomarker data for identifying mild cognitive impairment of the user based on first gaze information acquired while performing the first task and the second task a preset number of times.

7. The method according to claim 6, further comprising:
calculating a score value by inputting the digital biomarker data into a pre-learned mild cognitive impairment identification model; and
determining whether mild cognitive impairment exists based on the score value.

8. The method according to claim 1, wherein the gaze information is generated by the device analyzing an image after the device receives the image including eyes of the user from the user terminal.

9. The method according to claim 1, wherein the gaze information is information received by the device from the user terminal, and is generated by the user terminal analyzing an image including eyes of the user.

10. The method according to claim 6, wherein the digital biomarker data comprises information on a number of times the user gazed at the third region, information on a number of times the user gazed at a region other than the third region, information on whether the user's gaze continues to stare at a specific point for the preset time, information on a time elapsed for the user's gaze to move while performing the second task, information on movement speed of the user's gaze and information on whether the user's gaze is accurately staring at the third region.

11. A computer program stored on a computer-readable storage medium, wherein the computer program, when executed on at least one processor of a device, performs processes of identifying mild cognitive impairment, the processes comprising:
performing a task of acquiring digital biomarker data for identifying mild cognitive impairment of a user of a user terminal;
performing a preliminary task to allow the user to check the task before performing the task; and
acquiring gaze information of the user in conjunction with performing the task and the preliminary task;
wherein the gaze information is used when determining displayed information in the preliminary task, and used when identifying mild cognitive impairment of the user in the task.

12. A device for identifying mild cognitive impairment, the device comprising:
a storage configured to store at least one program instruction; and
at least one processor configured to execute the at least one program instruction,
wherein the at least one processor performs a task of acquiring digital biomarker data for identifying mild cognitive impairment of a user of a user terminal, performs a preliminary task to allow the user to check the task before performing the task, and acquires gaze information of the user in conjunction with performing the task and the preliminary task, and
wherein the gaze information is used when determining displayed information in the preliminary task, and used when identifying mild cognitive impairment of the user in the task.
